(19) United States
(12) Patent Application Publication
(10) Pub. No.: US 2002/0178504 A1
(43) Pub. Date: Dec. 5, 2002

(54) Body sleeping position detection for pillow height control that minimizes stress on neck and shoulder

(76) Inventor: Siu Ming Yung, Hong Kong (HK)

Correspondence Address: SIU MING YUNG, INTERNATIONAL INDUSTRIAL CENTRE, 18TH FLOOR, BLOCK X, 2-8 KWEI TEI STREET, FOTAN, HK

(21) Appl. No.: 09/873,891

(22) Filed: Jun. 1, 2001

(51) Int. Cl. A61B 5/103; A47C 27/10

(52) U.S. Cl. 5/713; 600/595; 5/600

(57) ABSTRACT

A pillow height control system includes a pillow height control device adapted to receive a body position signal that is generated by the body sleeping position sensor. The pillow control device is placed between the bed and the cushioning material that supports the sleeper's head. The device controls an electromechanical device that moves a platform that supports the cushioning material up or down to a position accurate enough to minimize the stress on the neck and shoulder. The fact that sleeper changes sleeping position from time to time several times during his sleep fails many designs. In preferred embodiments, user can adjust the optimum heights of the pillow for different sleeping positions.

### FIELD OF THE INVENTION

The present invention relates to a pillow height control system and more specifically to a pillow height control system which adjust the height of a pillow according to the body sleeping position accurate enough for minimizing the stress on the neck and shoulder.

### BACKGROUND OF THE INVENTION

A human body while sleeping has two major equilibrium positions. The first one is the symmetric position where the clavicle and scapula are in their horizontal positions. The second one is asymmetric position where the clavicle and scapula are in their vertical positions.

For the symmetric position, stress on the vertebra will be of minimum if the height of the pillow that supports the skull is so adjusted to have the neck placed horizontally.

For the asymmetric position, the height of the pillow that supports the skull has more effect on the stress on the vertebra, scapula as well as the shoulder muscle. If the pillow is too high, the vertebra stress is high; if the pillow is too low stresses on the vertebra, scapula as well as the shoulder muscle is higher.

Many attempts have been tried to minimize the stress. The first type of pillow design is to have pillow height optimized for either the symmetric or asymmetric position. The second type of pillow design is to have the pillow height in the middle portion optimized to suite the symmetric position, while the height in the left and right portions of the pillow is adjusted to the asymmetric position. The second type of pillow design assumes that the head will rotate to the left or right position. The second design fails to minimize the stress if the sleeper is in the asymmetric position while putting his head in the middle of the pillow.

Sleeper changes sleeping position from time to time several times during his sleep. Such designs described above offer improvements, but not practical to the actual situation.

Actually, the height of the pillow should change according to sleeping position; the pillow should be higher if the sleeper is in the asymmetric position, and the height should be decreased if the sleeper is in the symmetric position.

An Object of the invention is therefore to provide a body sleeping position detection such that the pillow height control that minimizes stress on neck and shoulder is possible.

### SUMMARY OF THE INVENTION

The invention provides a pillow height control system including a pillow height control device adapted to receive the body position signal that is generated by the body sleeping position detection device, wherein the body sleeping position detection device detects the symmetric or asymmetric sleeping position of the sleeper. The body sleeping position detection device generates the body position signal by detecting the difference in physical quantity while the sleeper are in the symmetric or asymmetric sleeping position, the said physical quantity may be pressure, capacitance, resistance or temperate which changes according to different the body sleeping positions.

Upon receiving the change in the body position signal, the said pillow height control device compares the platform height that is detected by a platform height sensor with the reference for optimum height and moves the said platform up or down to the desired height for that body position.

The said pillow height control device is an electromechanical device that can move a platform up and down. The pillow height control device is placed between the bed and the cushioning material that supports the sleeper's head, the cushioning material sits on top of the said platform so that by adjusting the height of the said platform effectively adjust the height of the pillow supporting the sleeper's head. Such electromechanical device includes hydraulic, mechanical machines such as gears box or level, and pressurized air bag systems.

The said platform height sensor may also produce digital on off signal that is generated by switches that are triggered when the said platform reaches the desired heights, the said on off signals represent the reference optimum low and high position of the said platform.

The said platform height sensor may produce an analog proportional signal generated by a variable resistance that couples to the movement of the said platform. The said analog proportional signal is the variable resistance physical quantity that varies according to the height of the said platform.

The said pillow height control device may be suitable for adapting existing pillow design so that the said device is placed inside a pillow.

In a simple embodiment, the desired positions can be controlled by positioning the platform height sensing switches at the positions correspond to the optimum heights for symmetric and asymmetric positions of the sleeper.

In some embodiments, the pillow height control device may store up the desired low and high pillow positions that correspond to the symmetric and asymmetric position of the sleeper, an external control unit can be used to adjust and store the pillow heights to a more satisfied position.

The said body sleeping position detection device is constructed to differentiate the symmetric position from the asymmetric position. The said detection device is a sensor that is placed parallel to the pillow and perpendicular to sleeper's vertebra.

Many physical properties can be employed for the detection of such positions. The area of the said sensor that is underneath one shoulder is smaller than the area of the said sensor that is underneath the back. The body temperate distribution along the said sensor is wider if the sleeper is in his symmetric position.

The said sensor can be embedded in the pillow if the thermal physical quantity is employed. The said sensor can also be an extension of the pillow that is placed underneath the shoulder of the sleeper if pressure, capacitance and resistance physical quantities are employed.

### BRIEF DESCRIPTION OF THE DRAWINGS

Embodiments of the invention will now be described by way of example only and with reference to the accompanying drawings, in which:

FIG. 1 shows a block diagram of the connection of the pillow height control system employing the present invention;

FIG. 2 shows the construction of first embodiment employing the present invention;

FIG. 2a shows the construction of the analog platform height sensor for first embodiment employing the present invention;

FIG. 3 shows the construction of the second embodiment employing the present invention;

FIG. 4a shows a diagram of the body sleeping position sensor that uses change of area physical quantity;

FIG. 4b shows a diagram of the body sleeping position sensor that uses temperature physical quantity.

### DETAILED DESCRIPTION OF THE PREFERRED EMBODIMENTS

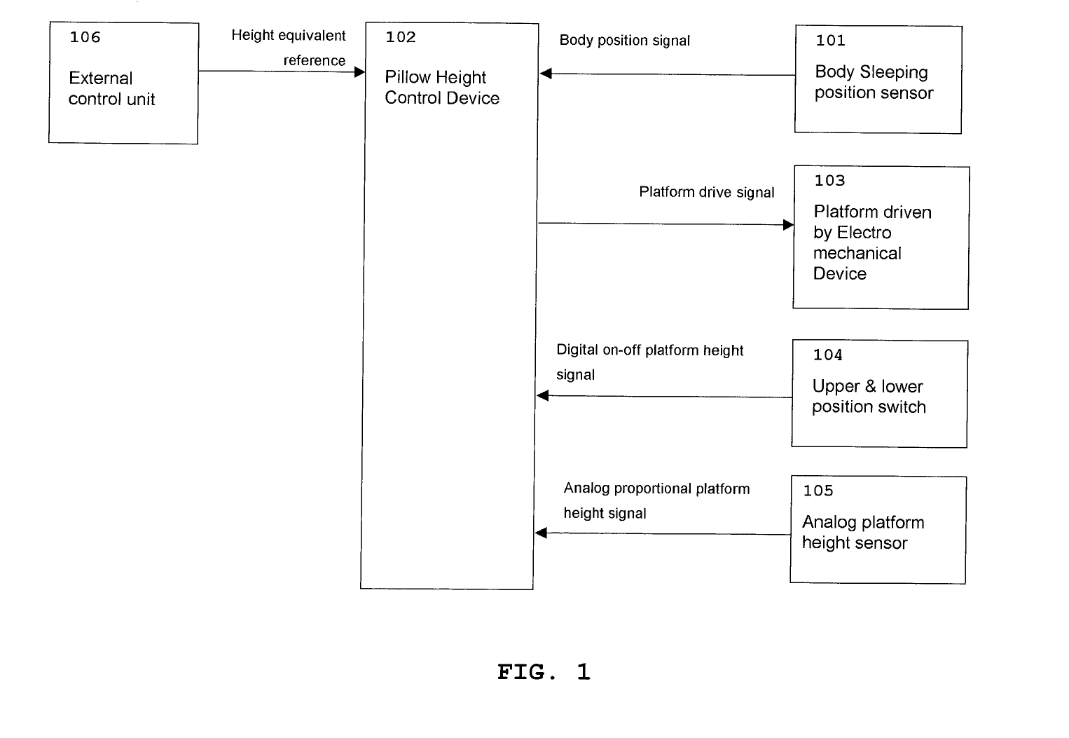

FIG. 1 shows the control of the first embodiment, a block diagram of the connection of the pillow height control system employing the present invention. The body position signal, which is generated by the body sleeping position sensor 101 measuring the body sleeping position, is connected to the pillow height control device 102. The said pillow height control device 102 compares the said body position signal with the desired pillow height for the said body sleeping position and drives the platform 103 that holds the cushioning material of the pillow up or down until the said platform 103 reaches the desired height.

The said desired height is detected by the upper and lower position switches 104 for logic on-off control, or the analog platform height sensor 105 for analog proportional control.

The said platform 103 can be driven by electromechanical device such as electric motor and gears assembly, if can also be driven by electromechanical device such as electric motor and air pump assembly. Any suitable device that can raise the said platform may be used.

The upper and lower position switches 104 govern the height of the said platform 103. Alternately, an analog platform height sensor 105 can also be used to adjust continuously the height to a desired position that is controlled by an external control device 106. Any suitable device that can control the height of the platform may be used.

The external control device 106 provides a mean to set the upper and lower height positions. The said external control device 106 provides height equivalent references for the said pillow height control device 102 to control the said platform 103 to the desired heights. If the analog position sensor 105 uses resistance as the height equivalent physical quantity, the said height equivalent references are the resistance physical quantities.

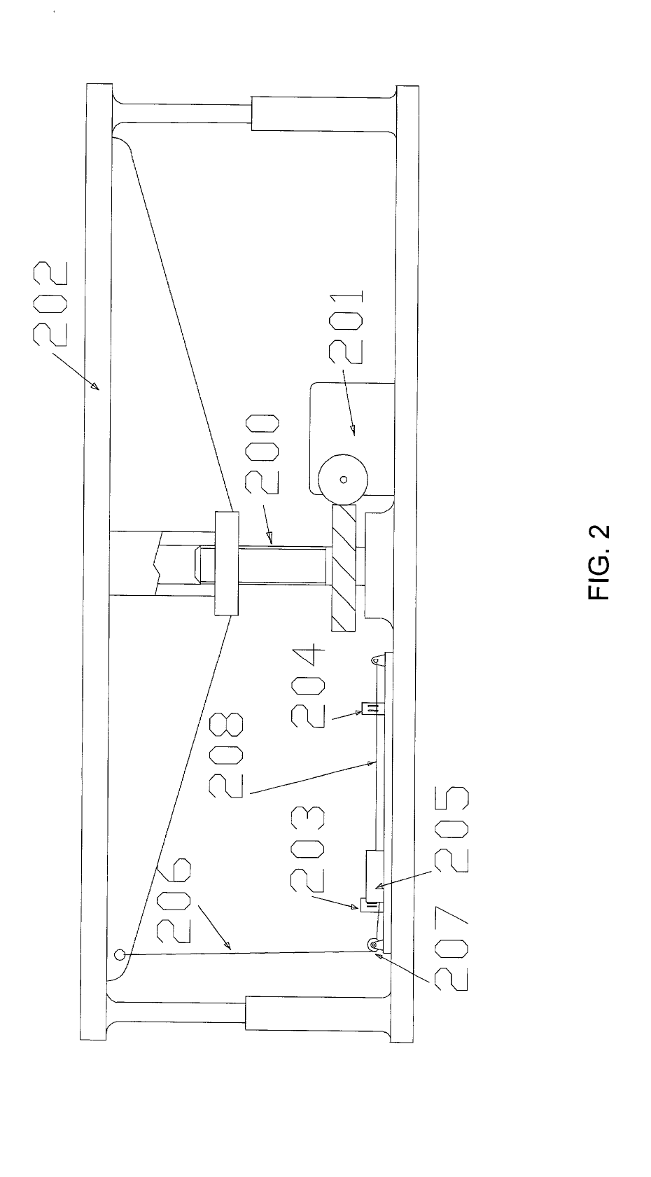

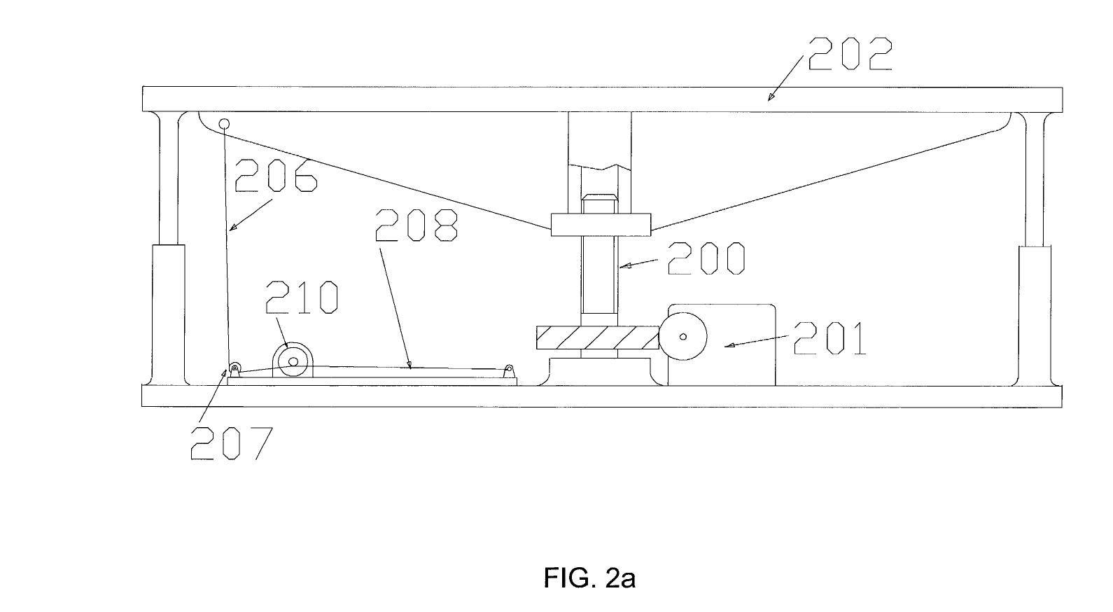

FIG. 2 shows the construction of first embodiment employing the present invention.

In this embodiment, A rigid box with a controllable height is employed such that it can be placed beneath a pillow, adjusting the height of the said rigid box effectively control the height of a pillow that supports the sleeper's head.

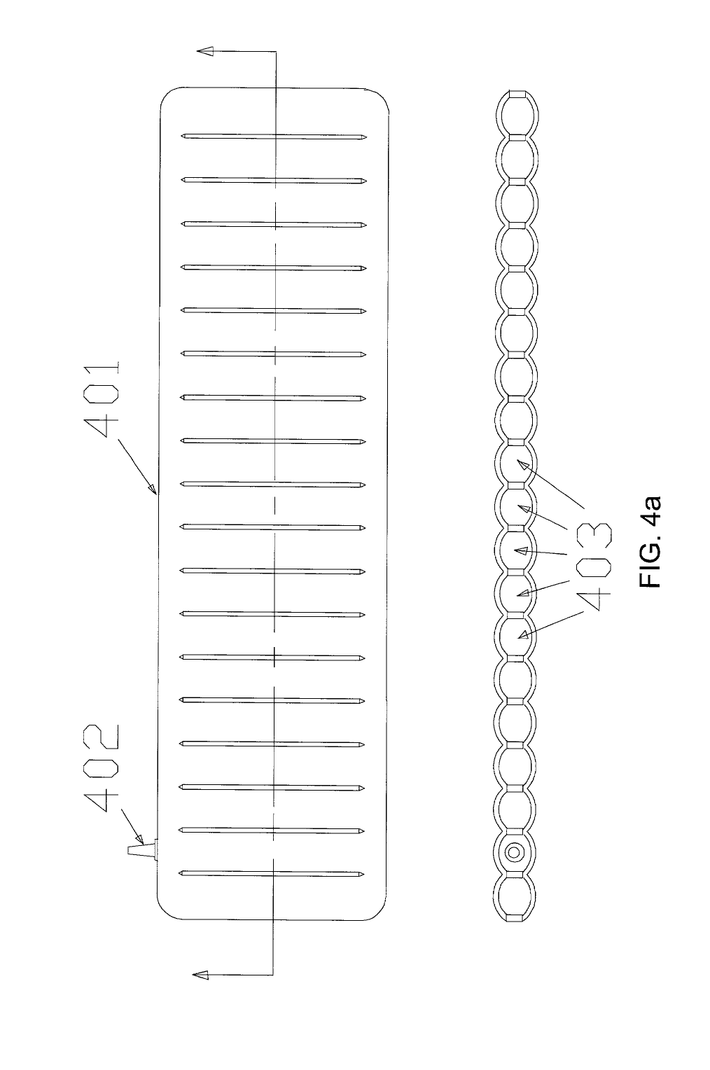

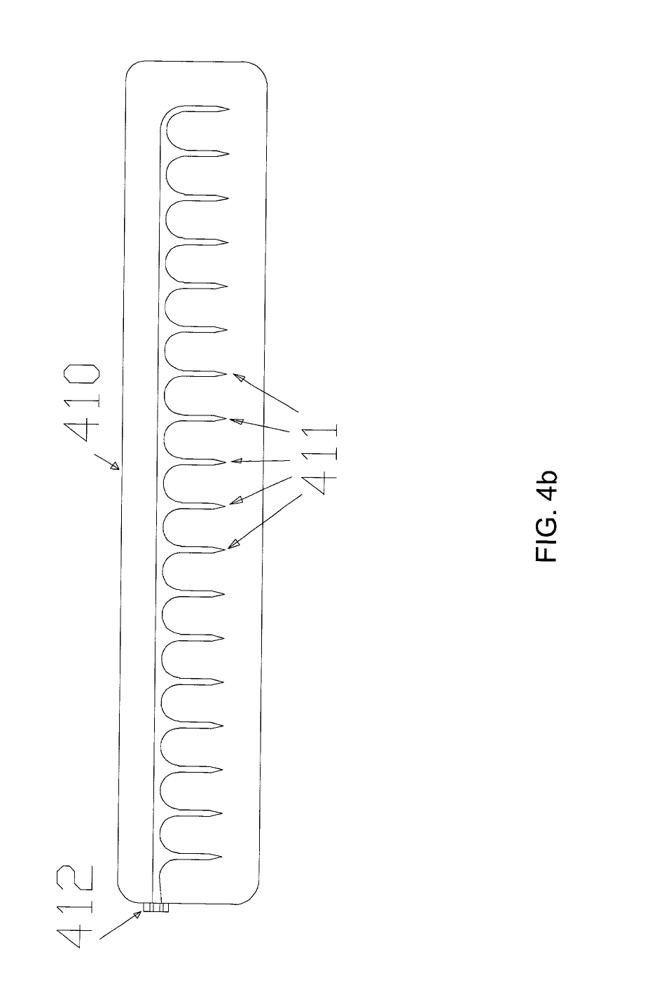

The body position signal from the body sleeping position sensor described in FIG. 4 is a logical signal indicates whether the sleeper is in the symmetric or asymmetric position. Upon receiving the signal, the pillow height control device drives the electromechanical device 201 in turn moves the platform 202 that holds the cushioning material supporting the sleeper's head to the desired height.

The electromechanical device is an electric motor gearbox assembly 201 that couples to the main screw 200. The electric motor turning in one direction raises the platform 202 while turning in the opposite direction lowers the platform 202.

The height of the said platform is reflected by the horizontal displacement of a block 205 that is connected to the top of the said platform 202 by a non-stretchable wire 206 via a pulley 207 that changes the axis of movement from vertical to horizontal. The other end of the said block 205 is connected to a fixture by a spring 208 that provides tension to the wire 206. The horizontal movement of the said block reflects the height of the said platform 202.

The upper position switch 203 and the lower position switch 204 are placed along the axis of movement of the said block 205. The said block 205 moves when the height of the said platform 202 changes, it triggers the upper position switch 203 or the lower position switch 204 when the position of the said block reaches the contact points of the said upper position switch 203 and lower position switch 204.

Setting the horizontal position of the upper position switch 203 and the lower position switch 204 governs the movement of the said platform 202 to the desired positions.

The lower position corresponds to the minimized stress position for sleeper in his symmetric sleeping position. The higher position corresponds to the minimized stress position for sleeper in his asymmetric sleeping position.

FIG. 2a shows the construction of the analog platform height sensor assembly for first embodiment employing the present invention.

The wire 206 is coupled to the moving arm of the variable resistance 210. For rotational variable resistance device, a few turns of wire 206 is wrapped around the rotating arms of the variable resistance. For linear variable resistance device, the wire 206 is attached to the linear moving arm of the variable resistance. The movement of the moving arms of the variable resistance 210 corresponds to the up or down movement of the said platform 202. The relative resistance of the said variable resistance 210 reflects the relative height of the said platform 202.

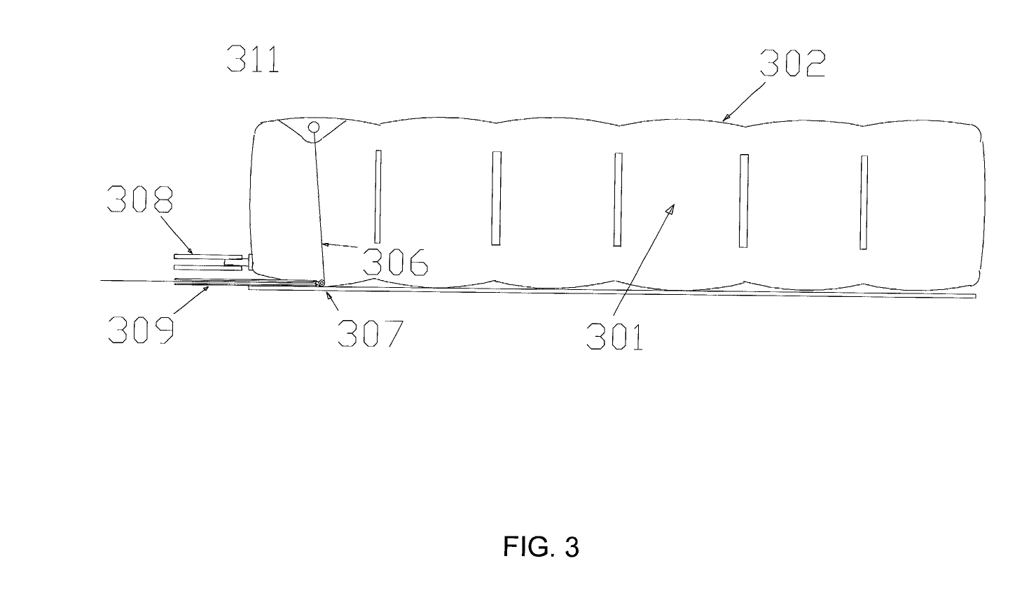

FIG. 3 shows the construction of the second embodiment employing the present invention.

In this embodiment, all the material used are soft and safety so that it is suitable for putting inside a pillow.

The electromechanical device is an electric air pump that pumps air into or out of the air bags 301 through the air tube 308. The air bags 301 consists of many equal compartments that are connected together, each compartment provides equal horizontal supports to the platform 302, which may be the upper body of the air bags 301 if a rigid cushioning material is used.

A non-stretchable wire 306 is connected to the top of the said platform 302, a pulley 307 changes the axis of movement of the wire 306 from vertical to horizontal.

The said wire 306 is extended to a platform height sensor assembly described in FIG. 2a through a fixed length conduit 309 such that the displacement of the said wire 306 relative to the conduit 309 reflects the height of the said platform.

The movement of the moving arms of the said variable resistance 210 as described in FIG. 2a corresponds to the up or down movement of the said platform 302. The relative resistance of the said variable resistance 310 reflects the relative height of the said platform 302.

The connections to the external device are an air tube 308 and a conduit 309, which can also be constructed by soft and safety materials. The platform height sensor assembly described in FIG. 2a and the air pump can be placed inside an external device.

FIG. 4a shows a diagram of the body sleeping position sensor that uses area physical quantity. The said body sleeping position sensor 401 is a bag with many interconnected compartments 403 of equal volume. Each compartment holds equal volume of air or fluid. The interconnections between compartments are permeable to air and fluid such that when the compartment 403 is totally compressed by the human body, the air or fluid contained in the compartment 403 is displaced to the next compartments and will not be blocked.

By placing the said body sleeping position sensor 401 parallel to the pillow and perpendicular to sleeper's vertebra, more compartments 403 will be compressed when the sleeper is in the symmetric sleeping position.

The said body sleeping positions sensor 401 allows using either air or fluid as the media to reflect the change in area physical quantity. If air is used, the change of air pressure measured at the nozzle 401 is proportional to the change in area. If fluid is used, the volume of the in-compressible fluid displaced from the nozzle 401 is proportional to the change in area.

FIG. 4b shows a diagram of the body sleeping position sensor that uses temperature physical quantity. The said body sleeping position sensor 410 is pad with many thermo-sensitive devices 411 connected in series.

The said thermo-sensitive device may be an array of thermocouples that develops voltage across the bi-metal junctions when exposed to different temperatures. The said change of voltage can be measured at the terminal 412. The change of voltage developed across the array of thermocouples connected in series is proportional to the change of area of the sensor that makes physical contact with the sleeper body.

The said thermo-sensitive devices may be an array of thermistors that changes its resistance when exposed to different temperatures. The said change of resistance can be measured at the terminal 412. The change of resistance across the array of thermistors connected in series is proportional to the change of area of the sensor that makes physical contact with the sleeper body.

What is claimed is:

1. An pillow height control system comprising a pillow height control device arranged to receive a body position signal from a body sleeping position sensor and a platform height signal from a platform height sensor, by comparing the platform height signal to a reference for optimum heights, said pillow height control device generates a platform driving signal to an electromechanical device, said electromechanical device drives a platform supporting the cushioning material of a pillow to a height that minimizes stress on neck and shoulder of a human body while resting the head on the pillow.

2. The pillow height control system of claim 1 wherein the said body sleeping position sensor generates a body position signal by detecting the change of physical quantity, the said physical quantity changes corresponding to the symmetric and asymmetric sleeping positions of a human body, said symmetric sleeping position corresponds to the sleeping position where the clavicle and scapula of a human body are in their horizontal positions, said asymmetric sleeping position corresponds to the sleeping position where the clavicle and scapula of a human body are in their vertical positions.

3. The pillow height control system of claim 1 wherein said electromechanical device receives the said platform driving signal, activates the mechanical force to raise or lower the said platform.

4. The pillow height control system of claim 1 wherein the said platform height is measured by a platform height sensor, the said platform height sensor provides physical quantity representing the height of the said platform.

5. The pillow height control system of claim 2 wherein the said body sleeping position sensor generates a body position signal by measuring the difference in area covered by the human body sleeping in symmetric and asymmetric positions.

6. The pillow height control system of claim 2 wherein the said body sleeping position sensor generates a body position signal by measuring the temperate distribution along the said sleeping position sensor for the human body sleeping in symmetric and asymmetric positions.

7. The pillow height control system of claim 2 wherein the said body sleeping position sensor generates a body position signal by measuring the difference in pressure for the human body sleeping in symmetric to asymmetric positions.

8. The pillow height control system of claim 3 wherein the said mechanical forces is supplied by pressurized air bag.

9. The pillow height control system of claim 3 wherein the said mechanical forces is supplied by hydraulic device.

10. The pillow height control system of claim 3 wherein the said mechanical forces is provided by mechanical machine.

11. The pillow height control system of claim 1 wherein the said pillow height control device accepts the said platform height signal generated by the said platform height sensor in a form of analog proportional signal.

12. The pillow height control system of claim 1 wherein the said pillow height control device accepts the said platform height signal generated by the said platform height sensor in a form of digital on off signal.

13. The pillow height control system of claim 1 wherein the said reference for optimum heights are provided by setting the positions of platform height sensors.

14. The pillow height control system of claim 1 wherein the said reference for optimum heights are provided by an external device.
